(19) United States
(12) Patent Application Publication
(10) Pub. No.: US 2023/0230693 A1
(43) Pub. Date: Jul. 20, 2023

(54) ARTIFICIAL INTELLIGENCE-BASED SYSTEM AND METHOD FOR REMOTE TREATMENT OF PATIENTS

(71) Applicant: Hsin-Yin Chiang, Strasbourg (FR)

(72) Inventor: Hsin-Yin Chiang, Strasbourg (FR)

(73) Assignee: Cephalgo SAS, Strasbourg (FR)

(21) Appl. No.: 18/096,112

(22) Filed: Jan. 12, 2023

Related U.S. Application Data
(60) Provisional application No. 63/301,479, filed on Jan. 20, 2022.

(51) Int. Cl. G16H 40/67; G16H 50/20; G16H 15/00; G16H 50/70; G16H 10/60

(52) U.S. Cl. CPC G16H 40/67; G16H 50/20; G16H 15/00; G16H 50/70; G16H 10/60

(57) ABSTRACT

The present invention discloses a system and a method for remote treatment of patients. The system comprises an acquisition device, a first device associated with the first user, a second device associated with a second user, and an artificial intelligence-based server. The system is further configured to execute one or more program modules: an input module configured to receive input data of the first user and enable the first user to input the feedback data, an analyser module configured to analyse the input data of the first user to diagnose the first user and suggest a first medical opinion, a report module configured to periodically generate and send a report to the second user. A suggestion module is configured to enable the second user to provide a second medical opinion to the first user, and enable the second user to modify the first medical opinion and the second medical opinion.

### CROSS-REFERENCE TO RELATED APPLICATION

The present application claims the benefit of U.S. provisional application 63/301,479 filed on 20 Jan. 2022 entitled “Remote Patient monitoring/Tracking System for Psychotherapy”, the contents of which are hereby incorporated by reference.

### BACKGROUND OF THE INNOVATION

### A. Technical Field

The present invention generally relates to system and method for remote treatment of patients, and, more particularly, to an artificial intelligence-based system and method for remote treatment of patients.

### B. Description of Related Art

Medical conditions related to, for example, psychology requires continual monitoring of patients to assure timely intervention by a healthcare practitioner or a physician to initiate the right medical procedure or administer the required medication in a timely manner Traditional method of patient tracking for psychotherapy involves visiting a human physician in-person, performing a series of tests to understand the patient's condition, receiving diagnosis and treatment plan from the physician. Further, to discuss the progress of the patient and the treatment plan, the patient needs to evaluate themselves and schedule follow-up with the physician.

These traditional methods of patient tracking for psychotherapy have their drawbacks. One drawback is the inconvenience of having to visit a physician in-person. Another drawback is the amount of time between each visit to the physician. The patients intentionally miss few months between each visit, when there is slight improvement in the health condition of the patient. Some patients are too weak to travel to the physician. Further some patients are incapable of discerning their progress to the treatment plan and may fail to visit the physician unintentionally, which will worsen the patient's condition. Furthermore, currently the feedback about psychotherapy highly relies on the patient's “feeling” which can be greatly influenced by memory-bias effects or overwritten by more recent feeling, failing to provide the objective and continuous patient tracking as the input for the next treatment planning Moreover, other pathologies like neurodegenerative diseases which lack objective biomarkers for identifying the symptoms require a better system of patient tracking to make progress in the investigation of treatments.

Therefore, there is a need for a system and method for remote treatment of patients. Further, there is a need for a system and method to remotely monitor the patients. Further, there is a need for a system and method to remotely diagnose and provide treatment plans to the patients. Further, there is a need for a system and a method to remotely monitor the progress of the patients undergoing the treatment plan to suggest further treatments or to modify the treatment plan.

### SUMMARY OF THE INNOVATION

The present invention discloses a system and a method for remote treatment of patients. In one embodiment, the system comprises, at least one biosignal acquisition device disposed at a vicinity proximal to a first user, the biosignal acquisition device is configured to measure biosignal data of the first user, a first device associated with the first user, and a second device associated with a second user, and an artificial intelligence-based server in communication with the first device and a second device. In one embodiment, the server comprises at least one memory unit for storing a set of program modules and a processor configured to execute the program modules. The server is configured to conduct patient-dependent analysis for personalized patient monitoring. In one embodiment, the biosignal acquisition device is a wearable device adapted to be worn by the first user.

Further, the modules comprise an input module, an analyser module, a report module, and a suggestion module. The input module is configured to receive input data of the first user and enable the first user to input the feedback data where the feedback data might be manually entered by the user or automatically measured while the user conducts designated tasks such as cognitive assessment exercises. The input data includes measurement data related to a biosignal of the first user, the feedback data of the first user. The input module is further configured to collect personalized biomarker from the first user and use the biomarker as the indicator for a treatment plan. In one embodiment, the analyser module is configured to analyse the input data of the first user to suggest at least one first medical opinion. The analyser module is configured to analyse the input data based on different groups of patient-related data. The groups are based on age, gender and medical history data. In one embodiment, the report module is configured to periodically generate and send a report to the second user. The report comprises the input data, data related to the analysis of the input data of the first user, data related to the diagnosis suggested by the AI-based server, data related to the first medical opinion suggested by the server. The medical opinion includes at least one of prognosis, diagnosis and treatment plan of a medical condition. A progress report including information related to the ongoing treatment plan, information related to the previous treatment plan and physiological and psychological response to the treatment plans. In one embodiment, a suggestion module is configured to enable the second user to provide a second medical opinion to the first user, and enable the second user to modify at least one of the first medical opinion and the second medical opinion.

In one embodiment, the analyser module is configured to correlate the input data of the first user to one or more health defects to diagnose the first user. The first user is a patient and the second user is a healthcare professional. The system further comprises a database in communication with the server to store information related to the first user, information related to the second user, information related to health defects and attributes of the health defects and information related medications and effects of the medications.

In some embodiments, the biosignal acquisition device includes at least one of an EEG system, ECG system, a functional near-infrared spectroscopy (fNIRS) system, a pulse oximeter, a sphygmomanometer and a thermometer. The system further comprises a learning module, which is configured to track the efficiency of each medical opinion, and classify the medical opinion that is least effective as ineffective opinion. Furthermore, the learning module is able to track the corresponding biosignal whenever the symptoms occur on the patients and further derive the biomarkers. The analyser module is configured to check if the medical opinion suggested by the server, and the second user is classified as ineffective opinion, and modify the medical opinion if the suggested plan is classified as ineffective opinion. The learning module is further configured to track progress of the patient with respect to each medical opinion and classify the opinion as effective opinions and ineffective opinions based on the progress of the patient. The system is configured to continuously learn and evolve from the classified medical opinion and treatment plan from a large group of patients.

In one embodiment, the input module is further configured to enable the second user to monitor the input data received from the first user. The input module is further configured to correlate the feedback data and the measurement data from the biosignal acquisition device to check the credibility of the first user. In one embodiment, the report module enables the second user to review, and edit the generated report, and enables the second user to approve, or reject the report. The report module is further configured to: send the report to the first user, and send the report to the first user after approval of the report by the second user.

According to another embodiment of the present invention, a method for remote treatment of patients, is disclosed. The method comprises: at one step, providing a system comprising: at least one biosignal acquisition device disposed at a vicinity proximal to a first user, the biosignal acquisition device is configured to measure biosignal data of the first user, a first device associated with the first user, a second device associated with a second user, wherein the first user is a patient and the second user is a healthcare professional, an artificial intelligence-based server in communication with the first device and a second device, the server comprises at least one memory unit for storing a set of program modules and a processor configured to execute the program modules, and a database in communication with the server to store information related to the first user, information related to the second user, information related to health defects and attributes of the health defects and information related medications and effects of the medications. The server is configured to conduct patient-dependent analysis for personalized patient monitoring. In one embodiment, the biosignal acquisition device is a wearable device adapted to be worn by the first user.

At another step, receiving, at an input module of the server, input data of the first user and enabling the first user to input the feedback data, the input data includes measurement data related to a biosignal of the first user, the feedback data of the first user, and personalized biomarker. At another step, analysing, at an analyser module of the server, the input data of the first user to diagnose the first user and suggest at least one first medical opinion. The analyser module is configured to analyse the input data based on different groups of patient-related data. The groups are based on age, gender and medical history data. At another step, periodically generating and sending, at a report module of the server, a report to the second user, the report comprises the input data, data related to the analysis of the input data of the first user, data related to the diagnosis suggested by the AI-based server, data related to the first medical opinion suggested by the server, and a progress report including information related to the ongoing treatment plan, information related to the previous treatment plan and physiological and psychological response to the treatment plans. Yet in another step, enabling, at a suggestion module of the server, the second user to provide a second medical opinion to the first user. Still, yet in another step, enabling, at the suggestion module of the server, the second user to modify at least one of the first medical opinion and the second medical opinion.

In one embodiment, the step of analysing involves correlating the input data of the first user to one or more health defects to diagnose the first user. The method further comprises a step of: tracking, at a learning module of the server, the efficiency of each medical opinion, classify the medical opinion that are least effective as ineffective opinion, and track the corresponding biosignal whenever the symptoms occur on the patients and further derive the biomarkers. In one embodiment, the step of analysing further involves: checking if the medical opinion provided by the server, and the second user is classified as ineffective opinion, and modify the medical opinion if the suggested medical opinion is classified as ineffective opinion.

In one embodiment, the method further comprises the step of, enabling, at the input module of the server, the second user to monitor the input data received from the first user. The method further comprises the step of, enabling, at the report module of the server, the second user to review, and edit the generated report, and to approve or reject the report. The method further comprises the step of, optionally sending, at the report module of the server, the report to the first user. The method further comprises the step of, optionally sending, at the report module of the server, the report to the first user after approval of the report by the second user.

Other objects, features and advantages of the present innovation will become apparent from the following detailed description. It should be understood, however, that the detailed description and the specific examples, while indicating specific embodiments of the innovation, are given by way of illustration only, since various changes and modifications within the spirit and scope of the innovation will become apparent to those skilled in the art from this detailed description.

### BRIEF DESCRIPTION OF DRAWINGS

The foregoing summary, as well as the following detailed description of the innovation, is better understood when read in conjunction with the appended drawings. For the purpose of illustrating the innovation, exemplary constructions of the innovation are shown in the drawings. However, the innovation is not limited to the specific methods and structures disclosed herein. The description of a method step or a structure referenced by a numeral in a drawing is applicable to the description of that method step or structure shown by that same numeral in any subsequent drawing herein.

FIG. 1 exemplarily illustrates an environment of a system for remote treatment of patients, according to an embodiment of the present invention.

FIG. 2 exemplarily illustrates components of the artificial intelligence based-server and the connection between the components of the system and the server, according to an embodiment of the present invention.

FIG. 3 exemplarily illustrates a flowchart of a method for remote treatment of patients, according to an embodiment of the present invention.

FIG. 4 exemplarily illustrates a flowchart for tracking and updating the efficiency of the treatment plan, according to an embodiment of the present invention.

FIG. 5 is a block diagram illustrating the operation of receiving feedback data, according to an embodiment of the present invention.

### DETAILED DESCRIPTION OF EMBODIMENTS

A description of embodiments of the present innovation will now be given with reference to the Figures. It is expected that the present innovation may be embodied in other specific forms without departing from its spirit or essential characteristics. The described embodiments are to be considered in all respects only as illustrative and not restrictive.

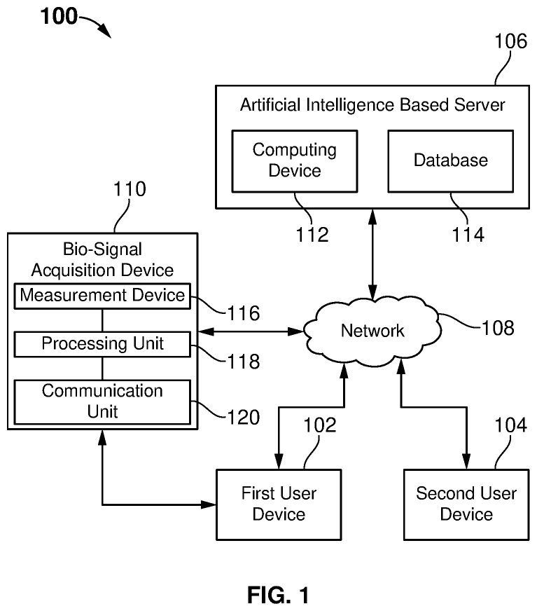

FIG. 1 exemplarily illustrates an environment 100 of a system for remote treatment of patients, according to an embodiment of the present invention. The system comprises an artificial intelligence-based server 106, a first user device 102, a second user device 104 and a biosignal acquisition device 110. The first user device 102 is associated with the first user and the second user device 104 is associated with the second user. In an example, the first user is a patient and the second user is a healthcare professional. The biosignal acquisition device 110 is disposed in a location proximal to the first user. The biosignal acquisition device 110, the first user device 102 and the second user device 104 are in communication with the artificial intelligence-based server 106 via a network 108. The artificial intelligence-based server 106 also referred as server 106 in this document. The term “first user” is also referred as patient and the term “second user” is also referred as healthcare professional. In an example, the system is configured for remote treatment including monitoring, diagnosis and tracking of patients for psychotherapy.

The biosignal acquisition device 110 is configured to measure biosignal of the first user and send the measurement data related to the biosignal of the first user to the server 106. In another embodiment, the biosignal acquisition device 110 is configured to send the measurement data to the first user device 102, which in turn sends the measurement data to the server 106. The device 110 comprises one or more measurement device 116, a processing unit 118 and a communication unit 120. The measurement device 116 is configured to measure the biosignal of the first user, and the processing unit 118 is configured to receive the measurement data for further processing and to send the measurement data to at least one of the server 106 and the first user device 102 via the communication unit 120. The server 106 is configured to conduct patient-dependent analysis for personalized patient monitoring. The measurement device 116 includes, but not limited to, electroencephalogram (EEG) sensors for measuring brainwaves, electrocardiogram (ECG) sensors for measuring heart rate, functional near-infrared spectroscopy (fNIRS) sensors to measure the concentration of haemoglobin in a patient's brain, sphygmomanometer for measuring blood pressure and thermometers or measuring body temperature. The biosignal acquisition device 110 is configured to send the measurement data in the form of non-transient, computer-readable media. The biosignal acquisition device 110 could include any measurement device 116 and sensors that are suitable to monitor and measure the biosignal of the patient and the medical condition of the patient. In one embodiment, the biosignal acquisition device 110 is a wearable device adapted to be worn by the first user.

The first user device 102 and the second user device 104 are a computing device configured to provide access to the service provided by the server 106. In an example, the first user is a patient and the second user is a healthcare professional. The user devices 102, 104 further have the capability to provide the user an interface to interact with the services provided by the server 106. The interface, for example, a mobile application that allows the device 102, 104 to wirelessly connect with the server 106 via the network 108. The user devices 102, 104 connect with the server 106 via Bluetooth by scanning a QR code. Further, the user devices 102, 104 could be connected with the server 106 via any other known methods. The user device 102, 104 may be, for example, a desktop computer, a laptop computer, a mobile phone, a personal digital assistant, and the like. The user device 102, 104 is configured to execute one or more client applications such as, without limitation, a web browser to access and view content over a computer network, an email client to send and retrieve emails, an instant messaging client for communicating with other users, and a File Transfer Protocol (FTP) client for file transfer. The user device 102, 104 in various embodiments, may include a Wireless Application Protocol (WAP) browser or other wireless or mobile device protocol suites.

The network 108 generally represents one or more interconnected networks, over which the first user device 102, the second user device 104, the biosignal acquisition device 110 and the server 106 can communicate with each other. The network 108 may include packet-based wide area networks (such as the Internet), local area networks (LAN), private networks, wireless networks, satellite networks, cellular networks, paging networks, and the like. A person skilled in the art will recognize that the network 108 may also be a combination of more than one type of network. For example, the network 108 may be a combination of a LAN and the Internet. In addition, the network 108 may be implemented as a wired network, or a wireless network or a combination thereof.

In one embodiment, the server 106 is at least one of a general or special purpose computer. In an embodiment, it operates as a single computer, which can be a hardware and/or software server, a workstation, a desktop, a laptop, a tablet, a mobile phone, a mainframe, a supercomputer, a server farm, and so forth. In an embodiment, the computer could be touchscreen and/or non-touchscreen device and could run on any type of OS, such as iOS™, Windows™, Android™, Unix™, Linux™ and/or others. In an embodiment, the computer is in communication with network 108. Such communication can be via a software application, a mobile app, a browser, an OS, and/or any combination thereof. The server 106 comprises a computing device 112 and at least one database 114. The server 106 is configured to store and process non-transient, computer-readable media.

In an embodiment, the database 114 may be accessible by the computing device 112. In another embodiment, the database 114 may be integrated into the server 106 or separate from it. In an embodiment, at least one database 114 resides in a connected server 106 or in a cloud computing service. In an embodiment, regardless of location, the database 114 comprises a memory to store and organize certain data for use by the server 106. In one embodiment, the database 114 stores information, including, but not limited to, health related data of the first user and the second user.

The computing device 112 is configured to receive input data of the first user. The computing device 112 is further configured to enable the first user to input the feedback data where the feedback data might be manually entered by the user or automatically measured while the user conducts designated tasks such as cognitive assessment exercises. The input data includes measurement data related to the biosignal of the first user and the feedback data of the first user. The computing device 112 is configured to check the credibility of the feedback data by correlating the feedback data from the patient with the measurement data of the biosignal of the first user. The computing device 112 is further configured to correlate the feedback data of the first user with the measurement data from the biosignal acquisition device 110 to check the credibility of the first user.

The computing device 112 is further configured to analyse the input data of the first user to diagnose the first user and suggest at least one first medical opinion. The medical opinion includes at least one of prognosis, diagnosis and treatment plan of a medical condition. The computing device 112 is configured to analyse the input data based on different groups of patient-related data. The groups are based on information, including, but not limited to, age, gender and medical history data. The computing device 112 is configured to correlate the input data of the first user to one or more health defects to diagnose the first user. The health defects, include, but not limited to, mental disorders, psychological disorders, psychological ailments, physical disorders, physical ailments, and chemical imbalances.

The computing device 112 is further configured to periodically generate and send a report to the second user. The report comprises the input data, data related to the analysis of the input data of the first user, data related to the diagnosis suggested by the AI-based server 106, data related to the first medical opinion suggested by the server 106, and a progress report including information related to the ongoing treatment plan, information related to the previous treatment plan and physiological and psychological response to the treatment plans. The computing device 112 is further configured to enable the second user to view the input data of the first user. The computing device 112 is further configured to enable the second user to review, and edit the generated report, and also enables the second user to approve or reject the report.

The computing device 112 is further configured to enable the second user to review the first medical opinion. The second user could either approve the first medical opinion, or reject the first medical opinion. The computing device 112 is further configured to enable the second user to modify the first medical opinion. The computing device 112 is configured to enable the second user to provide a new medical opinion, for example, a second medical opinion to the first user. The computing device 112 is configured to send or display the medical opinion approved by the second user. The patient is enabled to view the medical opinion by accessing the server 106 via the first user device 102. In another embodiment, the computing device 112 is configured to send the medical opinion and report of the patient to the first user device 102 associated with the patient.

The computing device 112 is configured to track the efficiency of each medical opinion of each medical condition, and classify the medical opinion that are least effective as ineffective or inefficient medical opinion. The computing device 112 is configured to track the corresponding biosignal whenever the symptoms occur on the patients and further derive the biomarkers. The computing device 112 is further configured to check if the medical opinion provided by the server 106, and the second user is classified as ineffective opinion, and modify the medical opinion if the suggested medical opinion is classified as ineffective opinion. The medical opinion includes at least one of prognosis, diagnosis and treatment plan of a medical condition. The computing device 112 is further configured to collect the personalized biomarker by demanding patient's feedback. The personalized biomarker could be used as the indicator for the treatment plan. The computing device 112 is configured to consider the diverse patients and develop the best treatment for each of them using AI. The system is configured to create a general artificial intelligence model and train the model, or algorithm for each patient according to their feedback. The system further uses the input from the health professionals to train the model. To avoid incoherent input from the patients, the patient feedback is reviewed for the coherence to ensure the system from corruption due to wrong input from patients. The computing device 112 is configured to enable the health professionals to supervise the results of the AI model and also provide inputs during the consultation or on behalf of patients as the main caregivers.

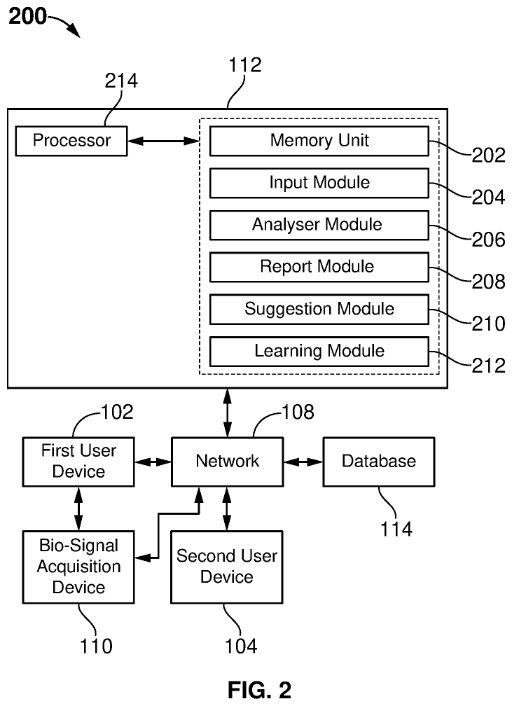

FIG. 2 exemplarily illustrates components of artificial intelligence-based server 106 and the connection between the components of the system and the server 106, according to an embodiment of the present. The server 106 comprises the computing device 112 and at least one database 114. The computing device 112 comprises a processor 214 and a memory unit 202. The memory unit 202 stores a set of program modules executable by the processor 214. The modules comprise an input module 204, an analyser module 206, a report module 208, a suggestion module 210 and a learning module 212. The input module 204 is configured to receive input data of the patient. The input data includes a measurement data related to the biosignal of the patient. The input module 204 is further configured to enable the first user to input a feedback data. The input data further includes feedback data. The feedback data comprises information related to feedback on the ongoing treatment plan of the patient. The feedback data further includes, but not limited to, information related to symptoms experienced by the patient, and information related to a current status of the patient.

The input module 204 is further configured to removes outliers in the manual patient feedback, and thus discredits or lowers the weight of a certain patient's feedback. The input module 204 further configured to prompt the healthcare professional to check the credibility of the feedback data from the patient. The database 114 comprises multitude of patient input measurements and patient feedback data (referred to herein as “initial data”). The initial data is gathered from a statistically significant number of patients in order to form a sample indicative of the possible population of patients. The initial data could be used to determine the credibility of the patient and the feedback data from the patient.

The analyser module 206 is configured to analyse the input data of the patient. The analyser module 206 is further configured to correlate the input data to health defects and provide diagnosis to the patient. The analyser module 206 is further configured to suggest a first medical opinion. The analyser module 206 is further configured to check if the first medical opinion provided by the server is classified as ineffective opinion, and modify the medical opinion if the suggested medical opinion is classified as ineffective opinion. The medical opinion includes at least one of prognosis, diagnosis and treatment plan of a medical condition. The analyser module 206 is configured to continuously analyse the input data of the patient. The system is configured to trigger an alarm if the patient exhibits unsafe qualities as determined by the system. For example, a patient with clinical depression may exhibit brain activity that corresponds with a greater potential for suicide. The system may make this determination and send an alarm to the healthcare professional in order to intervene and help the patient. The analyser module 206 is further configured to analyse the input data based on different groups of patient-related data. The groups are based on age, gender and medical history data. The analyser module 206 constantly learns and improves with the increasing amount of data of patients.

The report module 208 is further configured to generate a report comprising the input data, data related to the analysis of the patient, data related to the diagnosis suggested by the AI-based server 106 and data related to the first medical opinion suggested by the server 106. The report module 208 is further configured to generate the report periodically. The report module 208 is further configured to enable the healthcare professional to review the report. In another embodiment, the report module 208 is further configured to enable the patient to review the report. The report module 208 is further configured to periodically send the report to the healthcare professional. The report module 208 is further configured to send the report to the patient. The report module 208 is further configured to send the report to the patient after approval by the healthcare professional. In another embodiment, the healthcare professional could view the input data of the patient via the input module 204. The report module 208 is further configured to display the report to the first user and the second user.

The report module 208 is further configured to enable the second user to review, and edit the generated report. The report module 208 is further configured to enable the second user to approve or reject the report. The report further enables the second user to determine if there are any side effects to the suggested treatment plans and medications.

The suggestion module 210 is configured to send the first medical opinion to the patient. In another embodiment, the suggestion module 210 is configured to send the first medical opinion to the patient after being reviewed by the healthcare professional. The suggestion module 210 is configured to enable the second user to modify the first medical opinion. The suggestion module 210 is further configured to enable the second user to provide the second medical opinion to the patient. The analyser module 206 is further configured to check if the second medical opinion provided by the healthcare professional is classified as ineffective opinion, and prompt the healthcare professional to modify the second medical opinion if the second medical opinion is classified as ineffective opinion. In one embodiment, the medical opinion, or the treatment plan is provided to the first user in a format that is easily understandable by the first user. In one embodiment, the treatment plan includes medicated and non-medicated treatment plan. The non-medicated treatment plan includes a series of physical and/or mental exercises for the patient to complete at certain times throughout the day in order to relieve the patient of their ailment or to improve the patient's health condition.

The input module 204 is configured to enable the first user to input feedback data regarding ineffective treatment plans. The learning module 212 is configured to track the efficiency of each treatment plan of each medical diagnosis, and classify the treatment plan that are least effective as ineffective treatment plan. Furthermore, the learning module 212 is configured to track the corresponding biosignal whenever the symptoms occur on the patients and further derive the biomarkers. The server 106 is configured to learn the treatment plans that are best for certain health conditions and ailments by use of patient feedback and pre-existing clinical data in the database 114.

For example, a patient wears an fNIR sensor throughout the day, the biosignal acquisition device 110 measures the concentration of hemoglobin in a patient's brain. For a regular period of time, the server 106 is configured to analyze the patient's hemoglobin readings and determines that the patient suffers from clinical depression. Based on the input data, the server 106 is configured to recommend a treatment plan of 30 minutes of meditation twice daily for 8 weeks (herein after referred as initial treatment plan). The patient follows this treatment plan but does not feel that their mental health has improved. The patient could manually provide feedback to the system that the suggested treatment plan was ineffective. The system then notes the feedback from the patient and suggests a different treatment plan to the healthcare professional and patient. Furthermore, when the system determines that subsequent patients suffer from clinical depression, the system will not use the initial treatment plan on the subsequent patients, as the system has learned that the initial treatment plan is ineffective at treating clinical depression.

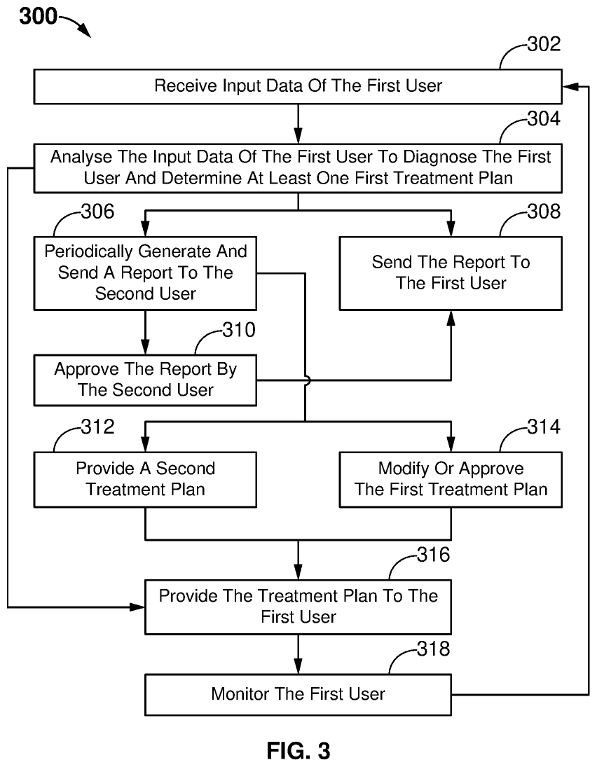

FIG. 3 exemplarily illustrates a flowchart of a method 300 for remote treatment of patients, according to an embodiment of the present invention. The method 300 is incorporated and executed by the system comprising the biosignal acquisition device 110, the first user device 102, the second user device 104, the artificial intelligence-based server 106 and the database 114. At step 302, the input module 204 receives input data of the first user. The input data includes a measurement data related to the biosignal of the patient. The input module 204 further enables the first user to input the feedback data. The input data further includes feedback data. The feedback data comprises information related to feedback on the ongoing treatment plan of the patient. The feedback data further includes information related to symptoms experienced by the patient. The input module 204 is further configured to collect personalized biomarker from the first user and use the biomarker as the indicator for the treatment plan.

The input module 204 further checks the credibility of the feedback data by correlating the feedback data and the designated assignment from the patient with the measurement data of the biosignal of the first user, as shown in FIG. 5. FIG. 5 is a block diagram 500 illustrating the operation of receiving feedback data, according to an embodiment of the present invention. The input module 204 further configured to correlate the feedback data of the first user device 102 and the designated assignment with the measurement data from the biosignal acquisition device 110 to check the credibility of the first user. For example, manual input data from the first user device 102 comprises a data “status 2” 502, and the measurement data from the device 110 comprises a data “status 1” 504. The “status 1” 504 is different from “the status 2” 502. Thus, the difference in the input data of this example could be used as a factor to check the credibility. Further, there may be also additional analysis to check the credibility.

At step 304, after receiving and checking the input data, the analyser module 206 analyses the input data of the first user to diagnose the first user and suggest at least one first medical opinion. The analyser module 206 is configured to analyse the input data based on different groups of patient-related data. The groups are based on age, gender and medical history data. The step 304 analyses the input data by correlating the input data of the first user to one or more health defects to diagnose the medical condition or health defects of the first user. At step 306, the report module 208 generates a report comprising the input data, data related to the analysis of the patient, data related to the diagnosis suggested by the AI-based server 106 and data related to the first medical opinion suggested by the server 106. At step 306, the report module 208 sends the report to the second user periodically. The report module 208 further enables the second user to review, and edit the generated report. The report module 208 further enables the second user to approve or reject the report.

At step 308, the report module 208 sends the report to the first user. In another embodiment, at step 310, the report needs to be approved by the second user to send the report to the first user.

At step 312, the suggestion module 210 enables the second user to provide the second medical opinion to the patient. At step 314, the suggestion module 210 enables the second user to modify or approve the first medical opinion. The second user could reject the first medical opinion and provide a new medical opinion, for example, the second medical opinion.

At step 316, the suggestion module 210 provides or sends the medical opinion to the first user. The medical opinion could be an opinion approved by the second user. In another embodiment, the suggestion module 210 could directly send the medical opinion to the first user.

At step 318, the first user is monitored via the input data received via the input module 204.

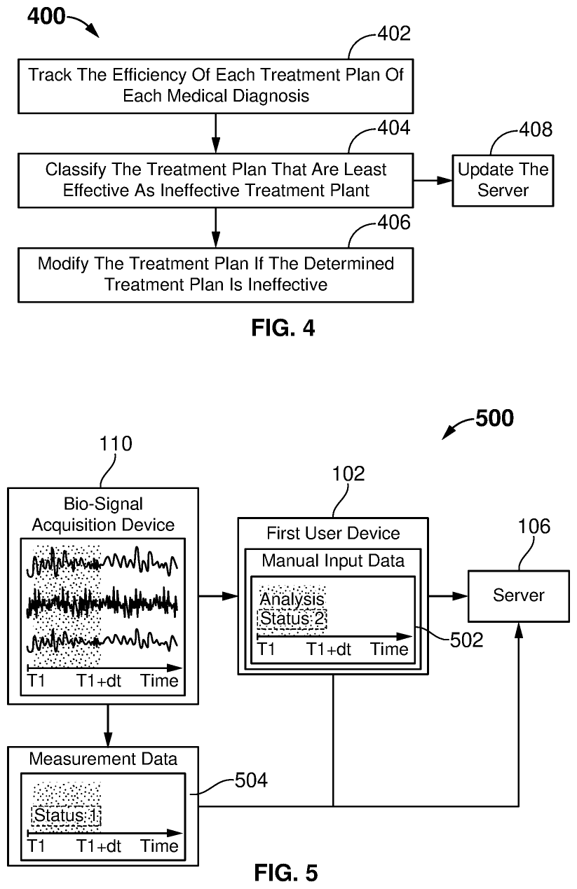

FIG. 4 exemplarily illustrates a flowchart 400 for tracking and updating the efficiency of the treatment plan, according to an embodiment of the present invention. According to this embodiment, the medical opinion includes treatment plan. At step 402, during monitoring, the server 106 and the healthcare professional could track the progress and responses of the first user to the treatment plan. At step 404, the learning module 212 classifies the treatment plan as ineffective or effective treatment plan based on the efficiency of the treatment plan. The learning module 212 tracks the corresponding biosignal whenever the symptoms occur on the patients and further derive the biomarkers. At step 406, by continuously tracking the progress, the server 106 and the healthcare professional could modify the treatment plan provided to the first user remotely. At step 408, the server 106 is updated according to the effectiveness of the treatment plans, including, but not limited to, the treatment plans that are least effective. Similarly, the system is configured to track and update progress of the patient with respect to each medical opinion and classify the opinion as effective opinions and ineffective opinions based on the progress of the patient. The medical opinion includes diagnosis, prognosis and treatment plan of a medical condition. The system is configured to continuously learn and evolve from the classified medical opinion and treatment plan from a large group of patients.

The system of the present invention provides reports regarding the patient at regular intervals so that the healthcare professional could continuously monitor the patient's well-being. Further, the system enables the healthcare professional to remotely diagnose the patient. The system further enables the patient to be aware of their health and the health defects. The AI capabilities of the invention would allow the system to improve its ability to provide accurate diagnoses and treatment plans after being subjected to a number of patient feedbacks. Furthermore, the system may use feedback from one patient to determine the diagnosis and treatment plan of another patient, thus taking feedback input from a large population of patients in order to compile more comprehensive feedback data.

Preferred embodiments of this innovation are described herein, including the best mode known to the innovators for carrying out the innovation. It should be understood that the illustrated embodiments are exemplary only and should not be taken as limiting the scope of the innovation.

The foregoing description comprises illustrative embodiments of the present innovation. Having thus described exemplary embodiments of the present innovation, it should be noted by those skilled in the art that the within disclosures are exemplary only, and that various other alternatives, adaptations, and modifications may be made within the scope of the present innovation. Merely listing or numbering the steps of a method in a certain order does not constitute any limitation on the order of the steps of that method. Many modifications and other embodiments of the innovation will come to mind to one skilled in the art to which this innovation pertains having the benefit of the teachings in the foregoing descriptions. Although specific terms may be employed herein, they are used only in generic and descriptive sense and not for purposes of limitation. Accordingly, the present innovation is not limited to the specific embodiments illustrated herein.

What is claimed is:

1. A system for remote treatment of patients, comprising:
at least one biosignal acquisition device adapted to be worn by a first user, the biosignal acquisition device is configured to measure biosignal data of the first user;
a first device associated with the first user;
a second device associated with a second user, and
an artificial intelligence-based server in communication with the first device and a second device, the server comprises at least one memory unit for storing a set of program modules and a processor configured to execute the program modules, the server is configured to conduct patient-dependent analysis for personalized patient monitoring, the modules comprise:
an input module configured to receive input data of the first user and enable the first user to input the feedback data, the input data includes measurement data related to a biosignal of the first user and the feedback data of the first user,
an analyser module configured to analyse the input data of the first user based on different groups of patient-related data, the groups are based on age, gender and medical history data, and
a report module configured to periodically generate and send a report related to the input data to the second user.

2. The system of claim 1, further comprises a suggestion module configured to enable the second user to provide a medical opinion to the first user.

3. The system of claim 2, wherein the suggestion module is configured to provide the medical opinion to the first user.

4. The system of claim 1, wherein the report comprises at least two of the following information including the input data, data related to the analysis of the input data of the first user, data related to the diagnosis by the AI-based server, data related to the medical opinion suggested by the server, a progress report including information related to the ongoing treatment plan, information related to the previous treatment plan, and physiological and psychological response to the treatment plans.

5. The system of claim 1, wherein the analyser module is configured to correlate the input data of the first user to one or more health defects to diagnose the first user.

6. The system of claim 1, wherein the first user is a patient and the second user is a healthcare professional.

7. The system of claim 1, further comprises a database in communication with the server to store information related to the first user, information related to the second user, information related to health defects and attributes of the health defects and information related medications and effects of the medications.

8. The system of claim 1, wherein the biosignal acquisition device includes at least one of an EEG system, ECG system, a functional near-infrared spectroscopy (fNIRS) system, a pulse oximeter sensor, a sphygmomanometer and a thermometer.

9. The system of claim 1, further comprises a learning module configured to track the efficiency of each medical opinion, classify the medical opinion that are least effective as ineffective medical opinion; and track a corresponding biosignal whenever the symptoms occur on the patients and derive the biomarkers.

10. The system of claim 1, wherein the analyser module is configured to check if the medical opinion provided by the server, and the second user is classified as ineffective medical opinion, and modify the medical opinion if the suggested medical opinion is classified as ineffective treatment plan.

11. The system of claim 1, wherein the input module is further configured to collect personalized biomarker from the first user and use the biomarker as the indicator for the treatment plan.

12. The system of claim 1, wherein the input module is further configured to enable the second user to monitor the input data received from the first user; and correlate the feedback data of the first user with the measurement data from the biosignal acquisition device to check the credibility of the first user.

13. The system of claim 1, wherein the report module: enables the second user to review, and edit the generated report enables the second user to approve or reject the report, send the report to the first user, and send the report to the first user after approval of the report by the second user.

14. A method for remote treatment of patients, comprising:
providing a system comprising:
at least one biosignal acquisition device adapted to be worn by a first user, the biosignal acquisition device is configured to measure biosignal data of the first user,
a first device associated with the first user,
a second device associated with a second user, wherein the first user is a patient and the second user is a healthcare professional,
an artificial intelligence-based server in communication with the first device and a second device, the server comprises at least one memory unit for storing a set of program modules and a processor configured to execute the program modules, the server is configured to conduct patient-dependent analysis for personalized patient monitoring, and
a database in communication with the server to store information related to the first user, information related to the second user, information related to health defects and attributes of the health defects and information related medications and effects of the medications;
receiving, at an input module of the server, input data of the first user and enable the first user to input the feedback data, the input data includes measurement data related to a biosignal of the first user, the feedback data of the first user and personalized biomarkers;
analysing, at an analyser module of the server, the input data of the first user using different groups of patient-related data, the groups are based on age, gender and medical history data;
periodically generating and sending, at a report module of the server, a report to the second user.

15. The method of claim 14, further comprising the steps of: enabling, at a suggestion module of the server, to provide a medical opinion to the first user;
enabling, at a suggestion module of the server, the second user to provide the medical opinion to the first user, and enabling, at the suggestion module of the server, the second user to modify the medical opinion.

16. The method of claim 14, wherein the report comprises the input data, data related to the analysis of the input data of the first user, data related to the diagnosis suggested by the AI-based server, data related to the medical opinion suggested by the server, and a progress report including information related to the ongoing treatment plan, information related to the previous treatment plan and physiological and psychological response to the treatment plans.

17. The method of claim 14, wherein the step of analysing involves correlating the input data of the first user to one or more health defects to diagnose the first user.

18. The method of claim 14, further comprises a step of: tracking, at a learning module of the server, the efficiency of each medical opinion, classify the medical opinion that are least effective as ineffective medical opinion, and track the corresponding biosignal whenever the symptoms occur on the patients and derive the biomarkers.

19. The method of claim 14, wherein the step of analysing further involves: checking if the medical opinion provided by the server, and the second user is classified as ineffective opinion, and modify the medical opinion if the suggested medical opinion is classified as ineffective opinion.

20. The method of claim 14, further comprising the step of: enabling, at the input module of the server, the second user to monitor the input data received from the first user;
enabling, at the report module of the server, the second user to review, and edit the generated report, and to at least one of approve, and reject the report;
optionally sending, at the report module of the server, the report to the first user, and
optionally sending, at the report module of the server, the report to the first user after approval of the report by the second user.
